(19) United States
(12) Patent Application Publication
(10) Pub. No.: US 2022/0361444 A1
(43) Pub. Date: Nov. 17, 2022

(54) LIVESTOCK MONITORING SYSTEM AND COMPONENTS THEREOF

(71) Applicant: Livestock Labs, Inc, Eveleigh (AU)

(72) Inventors: Timothy CANNON, Clinton, PA (US); Marlo WEBBER, Dundas Valley (AU); Amanda PLIMPTON, Carnegie, PA (US); Jessica WALDRIP, Oakdale, PA (US)

(73) Assignee: LIVESTOCK LABS, INC., Eveleigh (AU)

(21) Appl. No.: 17/309,307

(22) PCT Filed: Nov. 13, 2020
(86) PCT No.: PCT/US2020/070784

§ 371 Date: May 18, 2021

Related U.S. Application Data
(63) Continuation of application No. 16/686,742, filed on Nov. 18, 2019, now abandoned.

(51) Int. Cl. A01K 11/00; A01K 29/00

(52) U.S. Cl. CPC A01K 11/008; A01K 29/005

(57) ABSTRACT

Implantable devices, methods, and systems for using the implanted devices to monitor cattle are described herein.

### CROSS-REFERENCE TO RELATED APPLICATIONS

This application claims priority from PCT/US2020/070784 entitled “Livestock Monitoring System and Components Thereof” filed Nov. 13, 2020, which claims priority from, PCT/US2019/62038 entitled “Livestock Monitoring System and Components Thereof” filed Nov. 18, 2019, U.S. Provisional No. 62/768,682 entitled “Livestock Monitoring System and Components Thereof” filed Nov. 16, 2018 and U.S. Provisional No. 62/771,530 entitled “Livestock System and Components Thereof” filed Nov. 26, 2018 the entireties of which are incorporated by reference in their entireties.

### GOVERNMENT INTERESTS

Not applicable

### PARTIES TO A JOINT RESEARCH AGREEMENT

Not applicable

### INCORPORATION OF MATERIAL ON COMPACT DISC

Not applicable

### BACKGROUND

A significant part of livestock management is the necessity to monitor and treat the livestock for disease and illness. For example, both beef cattle and milk cattle are at risk to become infected with one or more of the various diseases occurring in animals.

Cattle like other livestock animals are prone to disease that causes several challenges. The first challenge is economic. Ill cattle for example requires additional special treatment, such as human labor for the separation and treatment, special equipment and medicine are also required. In some cases, the disease will end in the animal's death, causing more economical loss. A second challenge caused by animal illness is the risk for zoonotic diseases or namely diseases that can be transmitted from animal to humans. Such diseases might lead not only to economic damage, but also lead to a hazard to human health.

Different diseases are caused by bacteria, virus or other agents. Cattle disease influences different systems in the organism such as the respiratory, digestive, reproductive, neurological or other systems and can be expressed by a large variety of symptoms. Such symptoms can include, but not limited to: coughing, nasal and eye discharge, salivation, depression, lack of appetite and dullness, as well as other symptoms. In addition, one of the more significant symptoms leading to a diagnosis of disease is animal's high fever.

As the disease goes undiagnosed and untreated, it causes greater damage to tissues and organs which might eventually become permanently damaged. If identified early enough, the disease may be treatable by various means, such as medications or other methods. An early identification of an ill herd member will allow an early onset of treatment and may, for example, lower the chances of contamination to other members in the herd, lead to less complications and tissue damage, lead to less chances for future disease relapses, and lower mortality rates.

Currently sick cattle are identified visually. The farm staff visually examines the livestock to check for any changes in the animal's appearance or behavior. However, visual scanning lacks an objective and clear parameter for illness identification. The subjective visual check might be false-negative, for example, the person scanning the livestock might miss a sick animal due to lack of experience in identifying the characteristic signs and symptoms, cattle attempt to hide or mask signs of disease, or other reasons. On the other hand, a false-positive event of disease identification might also occur. Such case might lead to a waste of different resources and unnecessary medication treatment to the animal. Also, visual identifiable symptoms might occur relatively late in the disease course beyond the point of recovery for the animal.

Therefore, there is a need for a system capable for early detection of sick cattle and efficient transmission of the information to the farm staff.

### SUMMARY OF THE INVENTION

Embodiments of the invention are directed to implants including a biocompatible housing encapsulating: a circuit board, a battery operably connected to the circuit board, a processor operably connected to the circuit board, memory operably connected to the processor, a transmitter operably connected to the circuit board, a geolocation device operably connected to the circuit board, an accelerometer operably connected to the circuit board, and one or more medical instruments operably connected to the circuit board. In some embodiments, the implant may further include a long range antenna, a short range antenna, a Bluetooth antenna, a radio antenna, and combinations thereof. In some embodiments, the implant may further include, for example, a battery, a digital clock, and combinations thereof.

The one or more medical instruments of such embodiments may include thermometers, pulse meters, blood oxygen level meters, and the like and combinations thereof, and in some embodiments, the memory may include software necessary for enabling various functions of the implant and each of the one or more medical instruments. In some embodiments the processor may be configured to receive incoming signals, send incoming data to memory for temporary storage, process the incoming signals, create outgoing messages containing, medical data collected by the one or more medical instruments, geolocation data collected by the geolocation device, movement data collected by the accelerometer, and combinations thereof. In certain embodiments, the memory may include information relating to an implanted animal selected from the group consisting of lineage, age, medical history, medications, pregnancy history, owner contact information, and combinations thereof.

In certain embodiments, the housing may be composed of a polymer that is compliant with USP 6, ISO 10993 standards, and in some embodiments, the housing may be coated with a polymer that provides an impermeable biological barrier and/or is compliant with USP 6, ISO 10993 standards.

In some embodiments, the implant may include a low-power, short range radio antenna configured to collect data and communicate with other implants, and in some embodiments, the implant may include a long-range antenna configured to send and receive real-time alerts and tracking information to and from a computer network.

Other embodiments include a system for monitoring a population of animals that includes a computer control system configured to monitor the population of animals, and a plurality of implants comprising a circuit board, a battery operably connected to the circuit board, a processor operably connected to the circuit board, memory operably connected to the processor, a transmitter operably connected to the circuit board, a geolocation device operably connected to the circuit board, an accelerometer operably connected to the circuit board, one or more medical instruments operably connected to the circuit board wherein each implant is implanted in an animal in the population of animals and each implant is operably connected to the computer system by a network. In some embodiments, the computer system may be configured to monitor location, temperature, pulse, blood oxygen levels, fat density, and combinations thereof.

In some embodiments, the network may be the world wide web (Internet), a telecommunications network, a data network, an internet, an extranet, an intranet in communication with the Internet, an extranet in communication with the Internet, or combinations thereof. In certain embodiments, the network may include one or more computer servers enabled for distributed computing. In some embodiments, the computer system may include a peer-to-peer network that enables each implant coupled to the computer system to act as a client or a server.

In certain embodiments, the system may include one or more routers or beacons having known geographical locations in electronic communication with one or more of the plurality of implants and the computer control system. In some embodiments, the system may include one or more remote computer systems, personal computers, slate or tablet PC's, telephones, Smartphones, personal digital assistants and combinations thereof in communication with the computer system via the network.

### DESCRIPTION OF THE DRAWINGS

Examples of the specific embodiments are illustrated in the accompanying drawings. While the invention will be described in conjunction with these specific embodiments, it will be understood that it is not intended to limit the invention to such specific embodiments. On the contrary, it is intended to cover alternatives, modifications, and equivalents as may be included within the spirit and scope of the invention. In the following description, numerous specific details are set forth in order to provide a thorough understanding of the present invention. The present invention may be practiced without some or all of these specific details. In other instances, well known process operations have not been described in details so as to not unnecessarily obscure the present invention.

FIG. 1A is an illustration of a top view of an implant encompassed by the invention. FIG. 1B is an illustration of a side view of an implant encompassed by the invention.

FIG. 2 is a block diagram showing components of an implant encompassed by the invention

FIG. 3 is a schematic showing a method for geolocation encompassed by the invention.

FIG. 4 is a schematic showing information transfer of embodiments of the invention.

FIG. 5 is a schematic of a computer control system that is programmed or otherwise configured to implement methods provided herein.

### DETAILED DESCRIPTION

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Where a range of values is provided, it is intended that each intervening value between the upper and lower limit of that range and any other stated or intervening value in that stated range is encompassed within the disclosure. For example, if a range of 1 μm to 8 μm is stated, 2 μm, 3 μm, 4 μm, 5 μm, 6 μm, and 7 μm are also intended to be explicitly disclosed, as well as the range of values greater than or equal to 1 μm and the range of values less than or equal to 8 μm.

All percentages, parts and ratios are based upon the total weight of the topical compositions and all measurements made are at about 25° C., unless otherwise specified.

The singular forms “a,” “an,” and “the” include plural referents unless the context clearly dictates otherwise. Thus, for example, reference to a “polymer” includes a single polymer as well as two or more of the same or different polymers; reference to an “excipient” includes a single excipient as well as two or more of the same or different excipients, and the like.

The word “about” when immediately preceding a numerical value means a range of plus or minus 10% of that value, e.g, “about 50” means 45 to 55, “about 25,000” means 22,500 to 27,500, etc, unless the context of the disclosure indicates otherwise, or is inconsistent with such an interpretation. For example, in a list of numerical values such as “about 49, about 50, about 55, “about 50” means a range extending to less than half the interval(s) between the preceding and subsequent values, e.g, more than 49.5 to less than 52.5. Furthermore, the phrases “less than about” a value or “greater than about” a value should be understood in view of the definition of the term “about” provided herein.

By hereby reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by hereby reserving the right to proviso out or exclude any individual substituents, analogs, compounds, ligands, structures, or groups thereof, or any members of a claimed group, less than the full measure of this disclosure can be claimed for any reason. Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

A significant part of livestock management is the necessity to monitor and treat the livestock for disease and illness. For example, both beef cattle and milk cattle are at risk to become infected with one or more of the various diseases occurring in animals.

Cattle like other livestock animals are prone to disease that causes several challenges. The first challenge is economic. Ill cattle for example requires additional special treatment, such as human labor for the separation and treatment, special equipment and medicine are also required. In some cases, the disease will end in the animal's death, causing more economical loss. A second challenge caused by animal illness is the risk for zoonotic diseases or namely diseases that can be transmitted from animal to humans. Such diseases might lead not only to economic damage, but also lead to a hazard to human health.

Different diseases are caused by bacteria, virus or other agents. Cattle disease influences different systems in the organism such as the respiratory, digestive, reproductive, neurological or other systems and can be expressed by a large variety of symptoms. Such symptoms can include, but not limited to: coughing, nasal and eye discharge, salivation, depression, lack of appetite and dullness, as well as other symptoms. In addition, one of the more significant symptoms leading to a diagnosis of disease is animal's high fever.

As the disease goes undiagnosed and untreated, it causes greater damage to tissues and organs which might eventually become permanently damaged. If identified early enough, the disease may be treatable by various means, such as medications or other methods. An early identification of an ill herd member will allow an early onset of treatment and may, for example, lower the chances of contamination to other members in the herd, lead to less complications and tissue damage, lead to less chances for future disease relapses, and lower mortality rates.

Currently sick cattle are identified visually. The farm staff visually examines the livestock to check for any changes in the animal's appearance or behavior. However, visual scanning lacks an objective and clear parameter for illness identification. The subjective visual check might be false-negative, for example, the person scanning the livestock might miss a sick animal due to lack of experience in identifying the characteristic signs and symptoms, cattle attempt to hide or mask signs of disease, or other reasons. On the other hand, a false-positive event of disease identification might also occur. Such case might lead to a waste of different resources and unnecessary medication treatment to the animal. Also, visual identifiable symptoms might occur relatively late in the disease course beyond the point of recovery for the animal.

Therefore, there is a need for a system capable for early detection of sick cattle and efficient transmission of the information to the farm staff.

Various embodiments of the invention are directed to implants that monitor the health and growth of livestock animals in real time. In some embodiments, monitoring health may include monitoring biological aspects of the animal that most affect the animal's value as an agricultural product such as, for example, vital signs, distress, pregnancy and oestrus, the animal's growth rate, body composition, and the like and various combinations thereof.

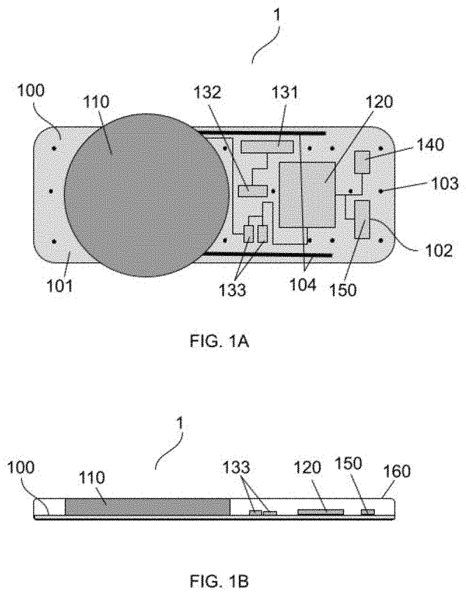

An example of an implant 1 of some embodiments is provided in FIG. 1A (upper surface) and FIG. 1B (side view). The implant 1 of embodiments may generally include a circuit board 100 that mechanically supports and electrically connects electronic and electrical components of the implant 1. The circuit board 100 may include various components necessary to electrically connect electronic and electrical components such as conductive tracks 101, pads 102, drill holes 103, antennae 104, and other features etched from sheets layers of copper laminated onto and/or between sheet layers of a non-conductive substrate. The one or more antennas 104 may be operably connected to an input and output terminal of the processor chip 120 either directly or via conductive tracks.

The implant 1 may further include a battery 110 that powers the device, and various other components electrically connected to the circuit board 100 such as, for example, central processor chip 120, transmitter 140, memory 150, and various other components 130. For example, the implant 1 of some embodiments may include a geolocation device 131, an accelerometer 132, and one or more medical instruments 133. The implant may further include other chips such as digital clocks. Medical instruments can include any medical instruments known in the art including for example thermometers, pulse meters, blood oxygen level meters, and the like and various combinations thereof. Such components may replace the components identified in FIG. 1 or be included in addition to the geolocation device 131, an accelerometer 132, and a thermometer 133. The specific build of these components will vary according to the implementation of the system, and it will be known to one of ordinary skill in the art how to configure them within the implant.

As more easily visualized in FIG. 1B the implant 1 may further include a housing or coating 160. For example, in some embodiments, the implant 1 may be coated in an polymer that provides bulk physical strength as well as an impermeable biological barrier. Such coating may generally meet standards for biocompatibility such as USP class 6, ISO 10993, and the like, and allow the implant 1 to be implanted under the skin of the livestock for the duration of the animal's life without leaking or reacting with biological tissues such as those associated with the immune system. In other embodiments, the implant 1 may be contained in a housing made of an epoxy or other material that provides bulk physical strength as well as an impermeable biological barrier. Such housing materials may meet standards for biocompatibility such as USP class 6, ISO 10993, and the like, like coating materials.

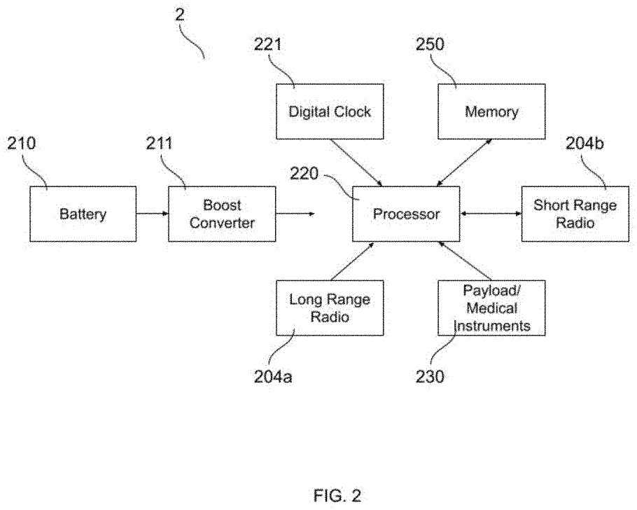

FIG. 2 is a block diagram showing movement of data within the implant 2 of FIG. 1. The battery 210 may be a single primary battery that stores enough power to operate for the animal's entire life. The battery 110 may be attached to the chip and can be connected to the device circuitry by vias or thin wires. However, in some cases, it is possible to use separate, prefabricated, component chips for memory, timing, processing, and demultiplexing. These are attached to the backside of the miniaturized delivery device with the battery.

In some embodiments, the battery 210 may be operably coupled to a boost converter 211. The battery 210 and boost converter 211 may provide all necessary power to the implant 2. The boost converter is used to convert the battery voltage, Vbat, to the compliance voltage V+. The compliance voltage V+ may provide power to the electrodes or other loads in implant 1 when, for example, the compliance voltage, V+, required is higher than the battery voltage, Vbat. For example, the battery voltage, Vbat, may be in the neighborhood of 4V, while compliance voltages of 18-20V may be necessary to provide sufficient current to power the implant or various components of the implant. The compliance voltage V+ can be adjusted depending on the power it must provide at any given time by the boost converter.

The central processor 220 may control all operations of implant 2. For example, in some embodiments, incoming signals may be received from long range radio antennas 204a and transferred to the processor 220 to be processed. The processor 220 may also send incoming data to memory 250 for temporary storage while the processor chip 220 processes the incoming signal. Outgoing messages containing, for example, medical data, geolocation data, or other data collected by the payload, which includes various, medical instruments 230 may be processed by the processor chip 220 and transmitted to a remote server or to a wireless computing device via the short range antennas 204b using, for example, Bluetooth technology, or long range radio antennas 204a. The antennas 204a,204b may be any type of antenna known in the art including long range antennas, short range antennas, Bluetooth antennas, radio antennas, and the like and combinations thereof. In some embodiments, a low-power, short range radio antenna 204b can be used for data collection and herd networking, and a more powerful long-range antenna 204a can be used for real-time alerts and tracking.

The memory 250 of the implant 2 may hold software necessary for enabling various functions of the implant 2 such as payload and medical instrument 230 functionality. For example, geolocation information the memory may provide instructions to take coordinates (e.g., latitude and longitude) or other location data, using a geolocation device payload 230, and store the location data in the memory 250 or transmit the location data to a remote server hosting a database using short range antennas 204b or long range radio antennas 204a. In some embodiments, an onboard digital clock 221 may time and date stamp the location data before storage or transmission.

In certain embodiments, the implant 2 may enter a sleep mode in which all or most payload and medical instrument 230 are stopped to limit power output from the battery 210. In such embodiments, power and function may be maintained for at least a digital clock 221 and, in some embodiments, a geolocation device, heart rate monitor chip, accelerometer chip payload 230, and the like and combinations thereof. In some embodiments, the digital clock 221 may trigger the processor chip to activate payload and medical instrument chips 230 at particular times during the day. For example, payload and medical instrument chips such as temperature chips, blood oxygen chips, heart rate monitor chips, geolocation device, and the like may be activated once per hour or every 4 hours, 6 hours, 12, hours, 24 hours, 48 hours, or any time period or range of time periods encompassed by these example time periods to acquire pertinent data which can be stored in memory 250 and transmitted to a remote server hosting a database using short range antennas 204b or long range radio antennas 204a.

In various embodiments, the implant 2 may be used for measuring biological aspects that most affect the animal's value as an agricultural product. For example, the implant 2 can be used to track an animals immediate vital signs and distress, pregnancy and oestrus, and the animal's growth rate and body composition.

In some embodiments, vital signs and distress alerts can be derived from pulse oximetry medical instrument 230. This instrument shines light into tissue at different wavelengths and measures how much of the light is reflected. As blood subtly changes color when oxygenated, the curves returned from this instrument show the animal's heartbeat, respiration, and blood pressure. Additionally, because muscle and fat are different colors, the pulse oximeter can also be used to indicate the body composition of the animal.

Pregnancy and oestrus are derived from very precise temperature measurements from a thermometer medical instrument 230, and in some embodiments, data derived from the thermometer can be supplemented by more advanced biochemical sensors. During oestrus, i.e. “heat,” female animals are slightly warmer than when the animal is not. Because this increase follows a recurring, predictable pattern, it is readily distinguishable from non-periodic increases such as fever, a precise thermometer can very accurately determine when the animal is in heat, and the system can identify the best time to breed the animal to maximize the probability that the animal will become pregnant. Although oestrus can be predicted accurately using a thermometer, other sensors that measure reproductive system hormones such as progesterone, which is correlated to both pregnancy and oestrus, can also be incorporated into the implants 2 of embodiments as payload/medical instruments 230.

The processor 220 can analyze raw measurement data onboard the implant 2 to create information-dense statistical data that can be stored in memory 250 and transmitted to a remote server hosting a database using short range antennas 204b or long range radio antennas 204a. Data can be retrieved from the implant by several different means, depending on the remoteness of the farm and what equipment is available. For example, in some embodiments, a networked herd of animals can be uplinked through a collar or tag worn by a lead animal, which can connect to cellular towers or a satellite. In some embodiments, data can be retrieved as animals pass a race gate or by transceivers at other points of common access such as a water trough or inside a truck. In some embodiments, data can be directly transmitted to a smartphone or data hub carried by the farmer or attached to machinery used to service the animals.

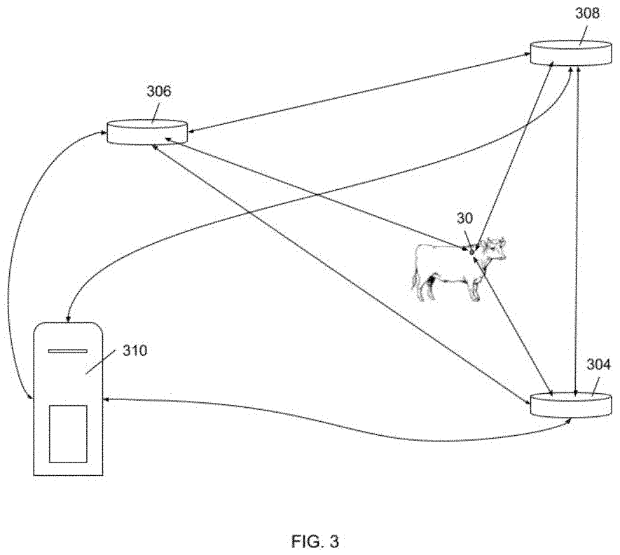

FIG. 3 illustrates how the location data can be obtained for the implant 30 when it is networked. In this example, one or more routers or beacons 304,306,308 having known geographical locations are used to obtain a distance to a networked implant 3. The geographical location of each router or beacon 304,306,308 may be configured during setup or otherwise ascertained with some precision. For example, router or beacon 304 may estimate its distance to the requesting network device based on the packet delay and the characteristics of the interface used to reach the network device (e.g., transmission speed of medium, type of physical medium: copper, fiber, wireless or others. etc.).

The networked implant 30 may request an IP address assignment from the communication network through which it communicates. In some embodiments, the latency from the network implant 301 may be small and not subject to large variations, and the router/beacon 304,306,308 can remove other uncertainty elements, like variable propagation delay and effects of transmission delay, from the latency estimate to make it more accurate.

Having obtained a distance of network device 30 to a router/beacon 304, a server/processor 310 may calculate a geographical location of the networked implant 30, using the geographical location of the router/beacon 304 and the distance to networked implant 30. Where other routers 306 and 308 having known geographical locations are available, the distance from each router is obtained and used to more precisely determine the geographical location of networked implant 30 by triangulation or other techniques.

In some embodiments, the networked implant 301 may have other mechanism, such as a GPS device, with which to obtain its own geographical location. Networked implant 301 can then include its geographical location when requesting an IP address from the communication network.

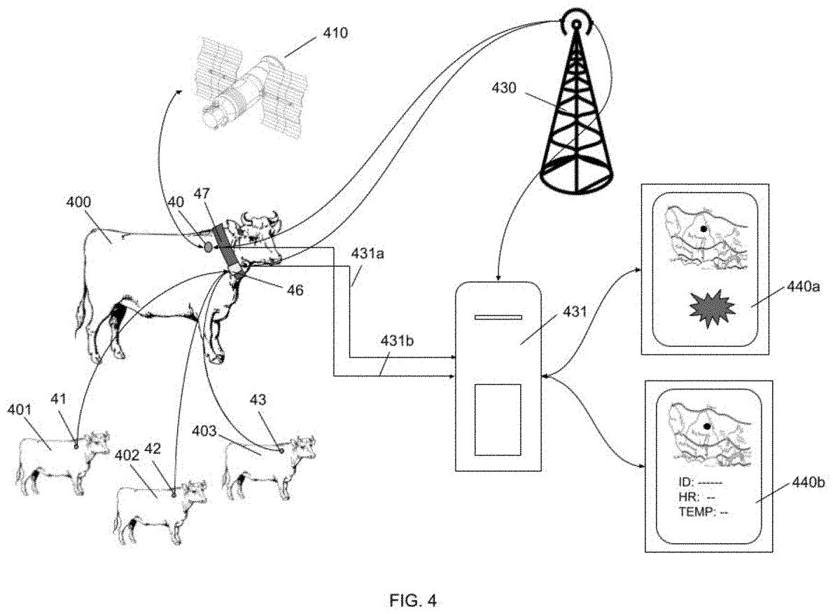

FIG. 4 shows a system diagram of some embodiments of the invention. As illustrated in FIG. 4 a series of cows 400,401,402,403 individually implanted with an implant 40,41,42,43 as described above. The implants 40,41,42,43 may connect to a server 420 directly using, for example, Bluetooth or radio transmissions 431 or indirectly through a satellite or cellular network 430. In some embodiments, each implant 40,41,42,43 may connect to the server individually via Bluetooth, radio transmissions 431a or a satellite or cellular network 430. In certain embodiments as illustrated in FIG. 4, each implant may communicate with an uplink implant or tag 46 shown, for illustrative purposes, attached to a collar 47 carried by a lead cow 400. The uplink implant or tag 46 may collect data from each cow of the herd 400,401,402,403 and transmit the data to the server 420 via Bluetooth, radio transmissions 431b, or a satellite or cellular network 430.

The system of FIG. 4 may provide a means for tracking animals locations and physical health. For example, a cow 400 with an implant 40 having a GPS chip may receive and send signals to a GPS satellites 410 orbiting the Earth. Using satellite signal responses the GPS receiver, the implant 40 can calculate the location of the animal and send this information to a server 420 directly using, for example, Bluetooth or radio transmissions 431 or indirectly through a satellite or cellular network 430. In various embodiments, the server 420 may perform comparisons of the location of the cow 400 against data entries for other implants 41,42,43 in other cows 401,402,403 of the herd. A cow 400 that is apart from the herd based on its GPS coordinates relative the other members of the herd, 401,402,403 may be identified as lost and the server may automatically signal a user through a user interface such as a smartphone, tablet computer, or other wireless computing device 440a, or via phone, email, text or the like. In other embodiments, the server may signal a user when an accelerometer on the implant 40 determines that the cow 400 is not moving, when the measured heart rate or temperature goes above or below a threshold set by a user, or combinations thereof. Such information may provide the user with information relating to the overall health of individual cows or the herd as a whole. For example, an increase in heart rate or temperature for an individual cow may indicate that the cow is distressed or ill, and a user may remove the cow from the herd to limit exposure of other cows to disease. An increase in heart rate or temperature of a number or cows in the heard may suggest the presence of a predator, and the user may take action to eliminate the threat.

The alert may be a visual, audible, tactile, and the like or combinations thereof. As illustrated in FIG. 4, in some embodiments, the alert may be a visual alert such as a blinking light and a map of the pasture showing the location of the separated cow. In certain embodiments the visual alert may be accompanied by an audible alert such as a sound, a tactile alert such as vibrating the device, and the like and combinations thereof.

In some embodiments, users can remotely connect with the server 420 via a software application user interface on a computing device 440. The user interface 440b may provide various information provided by the server 420 relating to individual animals 400,401,402,403 such as, for example, temperature updates, heart rate updates, and other health information and animal location updates, animal proximity to location and other animals of the herd. For example, in some embodiments, the software may provide a map of the tracked animal's current location, along with a historical location tracking list for a set period of time. In some embodiments, the software may provide real time health information for each cow of the herd.

In addition to remote monitoring of animal location and health information, users can wirelessly connect with the implant device by pairing it with a wireless computing device 440. Information such as lineage, age, medical history, medications, pregnancy history, owner contact information, and any other pertinent information can be stored in the memory of the implant and can be accessed through a software application the wireless computing device 440. A wireless connection can be created using, for example, Bluetooth, wife, RFID, radio transmitters, and the like and may provide historical information to the user that can be compared to real time or recent data to determine the health of the animal. In some embodiments, a veterinarian or other animal health caregiver may add health information to the implants memory from the wireless computing device that can be accessed at a later date. In some embodiments, the veterinarian may provide authentication with the added health information.

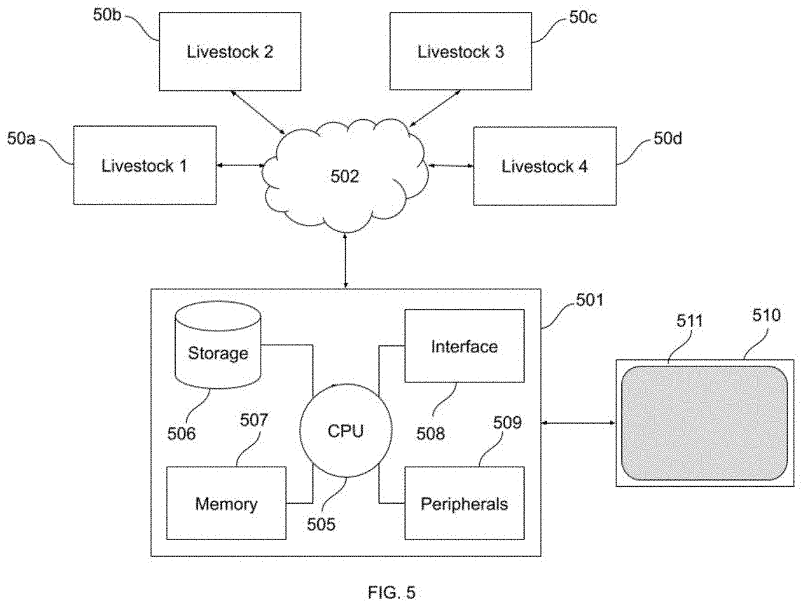

Further embodiments include computer control systems that are programmed to implement the various methods described above. FIG. 5 shows a computer system 501 that is programmed or otherwise configured to monitor a population of animals, for example, a herd of cattle. The cows 50a,50b,50c,50d may be connected to computer system 501 through network 502. The computer system 501 can monitor the cows 50a,50b,50c,50d, such as, for example, monitoring location, temperature, pulse, blood oxygen levels, fat density, and the like and combinations thereof. In some embodiments, methods include monitoring the cows, analyzing the cows data, and alerting a user of changes in the health or location of the cows.

The computer system 501 may include a central processing unit (“CPU,” “processor,” or “computer processor”) 505, and the CPU 505 can be a single core or multi core processor, or a plurality of processors for parallel processing. The computer system 501 may also include memory 506 (e.g., random-access memory, read-only memory, flash memory), electronic storage unit 507 (e.g., hard disk), communication interface 508 (e.g., network adapter) for communicating with one or more other systems, and peripheral devices 509, such as cache, other memory, data storage and/or electronic display adapters. The memory 506, storage unit 507, interface 508 and peripheral devices 509 may be in communication with the CPU 505 through a communication bus (solid lines), such as a motherboard. The storage unit 507 can be a data storage unit (or data repository) for storing data.

The computer system 501 can be operatively coupled to a computer network (“network”) 502 with the aid of the communication interface 508. The network 502 can be the Internet, an internet and/or extranet, or an intranet and/or extranet that is in communication with the Internet. The network 502 in some cases is a telecommunication and/or data network. The network 502 can include one or more computer servers, which can enable distributed computing, such as cloud computing. The network 502, in some cases with the aid of the computer system 501, can implement a peer-to-peer network, which may enable devices coupled to the computer system 501 to behave as a client or a server.

The CPU 505 can execute a sequence of machine-readable instructions, which can be embodied in a program or software. The instructions may be stored in a memory location, such as the memory 506. The instructions can be directed to the CPU 505, which can subsequently program or otherwise configure the CPU 505 to implement methods of the present disclosure. Examples of operations performed by the CPU 505 can include fetch, decode, execute, and writeback. The CPU 505 can be part of a circuit, such as an integrated circuit. One or more other components of the system 501 can be included in the circuit. In some cases, the circuit is an application specific integrated circuit (ASIC).

The storage unit 507 can store files, such as drivers, libraries and saved programs. The storage unit 507 can store user data, e.g., user preferences and user programs. The computer system 501 in some cases can include one or more additional data storage units that are external to the computer system 501, such as located on a remote server that is in communication with the computer system 501 through an intranet or the Internet.

The computer system 501 can communicate with one or more remote computer systems through the network 502. For example, the computer system 501 can communicate with a remote computer systems such as personal computers, slate or tablet PC's, telephones, Smartphones, or personal digital assistants. The user can access the computer system 501 via the network 502.

Methods as described herein can be implemented by way of machine (e.g., computer processor) executable code stored on an electronic storage location of the computer system 501, such as, for example, on the memory 506 or electronic storage unit 507. The machine executable or machine readable code can be provided in the form of software. During use, the code can be executed by the processor 505. In some cases, the code can be retrieved from the storage unit 507 and stored on the memory 506 for ready access by the processor 505. In some situations, the electronic storage unit 507 can be precluded, and machine-executable instructions are stored on memory 506.

The code can be pre-compiled and configured for use with a machine have a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 501, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 501 can include or be in communication with an electronic display 510 that comprises a user interface (UI) 511 for providing, for example, the ability to monitor and/or regulate multiple robotic greenhouse systems at the same time and/or from one user interface. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 505. The algorithm can, for example, monitor health of a cow; receiving an indication of the cows overall health or growth; associate the growth characteristic with a stage of growth of the given plant; and modify growing conditions of the given plant based on the determined stage of growth of the given plant.

1. An implant comprising a biocompatible housing encapsulating:
a circuit board;
a battery operably connected to the circuit board;
a processor operably connected to the circuit board;
memory operably connected to the processor;
a transmitter operably connected to the circuit board;
a geolocation device operably connected to the circuit board;
an accelerometer operably connected to the circuit board;
one or more medical instruments operably connected to the circuit board.

2. The implant of claim 1, further comprising a long range antenna, a short range antenna, a Bluetooth antenna, a radio antenna, and combinations thereof.

3. The implant of claim 1, further comprising a battery.

4. The implant of claim 1, further comprising a digital clock.

5. The implant of claim 1, wherein the one or more medical instruments are selected from the group consisting of thermometers, pulse meters, blood oxygen level meters, and combinations thereof.

6. The implant of claim 1, wherein the housing is coated with a polymer that provides an impermeable biological barrier.

7. The implant of claim 6, wherein the coating is compliant with USP 6, ISO 10993 standards.

8. The implant of claim 1, wherein the housing is compliant with USP 6, ISO 10993 standards.

9. The implant of claim 1, wherein the processor is configured to receive incoming signals, send incoming data to memory for temporary storage, process the incoming signals, create outgoing messages containing, medical data collected by the one or more medical instruments, geolocation data collected by the geolocation device, movement data collected by the accelerometer, and combinations thereof.

10. The implant of claim 1, wherein the memory comprises software necessary for enabling various functions of the implant and each of the one or more medical instruments.

11. The implant of claim 1, wherein the memory comprises information relating to an implanted animal selected from the group consisting of lineage, age, medical history, medications, pregnancy history, owner contact information, and combinations thereof.

12. The implant of claim 1, further comprising a low-power, short range radio antenna configured to collect data and communicate with other implants.

13. The implant of claim 1, further comprising a long-range antenna configured to send and receive real-time alerts and tracking information to and from a computer network.

14. A system for monitoring a population of animals comprising:
a computer control systems configured to monitor the population of animals;
a plurality of implants comprising a circuit board, a battery operably connected to the circuit board, a processor operably connected to the circuit board, memory operably connected to the processor, a transmitter operably connected to the circuit board, a geolocation device operably connected to the circuit board, an accelerometer operably connected to the circuit board, one or more medical instruments operably connected to the circuit board;
wherein each implant is implanted in an animal in the population of animals and each implant is operably connected to the computer system by a network.

15. The system of claim 14, wherein the computer system is configured to monitor location, temperature, pulse, blood oxygen levels, fat density, and combinations thereof.

16. The system of claim 14, the network is selected from the group consisting of the word wide web (Internet), a telecommunications network, a data network, an internet, an extranet, an intranet in communication with the Internet, an extranet in communication with the Internet, and combinations thereof.

17. The system of claim 14, wherein the network comprises one or more computer servers enabled for distributed computing.

18. The system of claim 14, wherein the computer system comprises a peer-to-peer network that enables each implant coupled to the computer system to act as a client or a server.

19. The system of claim 14, further comprising one or more routers or beacons having known geographical locations in electronic communication with one or more of the plurality of implants and the computer control system.

20. The system of claim 14, further comprising one or more remote computer systems, personal computers, slate or tablet PC's, telephones, Smartphones, personal digital assistants and combinations thereof in communication with the computer system via the network.
